(19) United States
(12) Patent Application Publication
(10) Pub. No.: US 2007/0173728 A1
(43) Pub. Date: Jul. 26, 2007

(54) Cyclic variation of heart rate detection and treatment

(76) Inventors: Yachuan Pu, Minneapolis, MN (US); Quan Ni, Shoreview, MN (US); Yinghong Yu, Shoreview, MN (US)

Correspondence Address: HOLLINGSWORTH & FUNK, LLC, Suite 125, 8009 34th Avenue South, Minneapolis, MN, 55425, US

(21) Appl. No.: 11/338,997

(22) Filed: Jan. 25, 2006

(51) Int. Cl. A61M 16/00; A61B 5/02; A61B 5/08; A62B 7/00

(52) U.S. Cl. 600/484; 600/529; 128/204.23

(57) ABSTRACT

Systems and methods provide for detection of repeated disordered breathing episodes and presence of cyclic variation of heart rate (CVHR) and/or associated presence of cyclic variations in hemodynamic changes resulting from such disordered breathing episodes. A therapy may be delivered to mitigate the CVHR. The therapy for CVHR mitigation or other therapy may be delivered or adjusted to mitigate the disordered breathing.

### FIELD OF THE INVENTION

The present invention relates generally to detection of disordered breathing and cyclic variation of heart rate resulting from such disordered breathing and, more particularly, to treatment of cyclic variation of heart rate.

### BACKGROUND OF THE INVENTION

Sleep is generally beneficial and restorative to a patient, exerting great influence on the quality of life. The human sleep/wake cycle generally conforms to a circadian rhythm that is regulated by a biological clock. Regular periods of sleep enable the body and mind to rejuvenate and rebuild. The body may perform various tasks during sleep, such as organizing long term memory, integrating new information, and renewing tissue and other body structures.

Sleep apnea is a fairly common breathing disorder characterized by periods of interrupted breathing experienced during sleep. Sleep apnea is typically classified based on its etiology. One type of sleep apnea, denoted obstructive sleep apnea, occurs when the patient's airway is obstructed by the collapse of soft tissue in the rear of the throat. Central sleep apnea is caused by a derangement of the central nervous system control of respiration. The patient ceases to breathe when control signals from the brain to the respiratory muscles are absent or interrupted. Mixed apnea is a combination of the central and obstructive apnea types. Regardless of the type of apnea, people experiencing an apnea event stop breathing for a period of time. The cessation of breathing may occur repeatedly during sleep, sometimes hundreds of times a night and occasionally for a minute or longer.

In addition to apnea, other types of disordered respiration have been identified, including, for example, hypopnea (shallow breathing), dyspnea (labored breathing), hyperpnea (deep breathing), and tachypnea (rapid breathing). Combinations of the disordered respiratory events described above have also been observed. For example, Cheyne-Stokes respiration (CSR) is associated with rhythmic increases and decreases in tidal volume caused by alternating periods of hyperpnea followed by apnea and/or hypopnea. The breathing interruptions of CSR may be associated with central apnea, or may be obstructive in nature. CSR is frequently observed in patients with heart failure (HF) and is associated with an increased risk of accelerated HF progression.

An adequate duration and quality of sleep is required to maintain physiological homeostasis. Untreated, sleep disorders may have a number of adverse health and quality of life consequences ranging from high blood pressure and other cardiovascular disorders.

### SUMMARY OF THE INVENTION

The present invention is directed to systems and methods for detecting disordered breathing episodes and associated presence of cyclic variations in hemodynamic changes resulting from such disordered breathing episodes.

According to an embodiment of the present invention, repeated occurrences of disordered breathing are detected. After detecting the repeated occurrences of disordered breathing, changes of a parameter indicative of cyclic variation of heart rate (CVHR), for example, are detected. Detection of the disordered breathing episodes typically involves detection of repeated occurrences of sleep disordered breathing events, such as repeated occurrences of apnea, hypopnea or other form of sleep disordered breathing. Detection of disordered breathing may be performed patient-internally, patient-externally, or a combination of both approaches. Detection of CVHR may be performed patient-internally, patient-externally, or a combination of both approaches.

Detecting repeated occurrences of sleep disordered breathing may involve detecting variations of a respiratory signal indicative of sleep disordered breathing. Detecting repeated occurrences of sleep disordered breathing may involve detecting variations of an electrocardiogram signal indicative of sleep disordered breathing.

Parameters that may be useful for detecting the presence of CVHR include heart rate, a surrogate signal for heart rate, blood gas saturation, blood pressure, a parameter indicative of patient hemodynamics, cardiac output, autonomic activity, intrathoracic pressure, and left ventricular transmural pressure, for example. Detecting changes of the parameter indicative of CVHR may involve determining a periodicity of the parameter changes. Detecting changes of the parameter indicative of CVHR may involve determining a measure of stability of the parameter changes.

According to another embodiment of the present invention, repeated occurrences of sleep disordered breathing are detected, and, after detecting the repeated occurrences of sleep disordered breathing, changes of a parameter indicative of CVHR are detected. A therapy is delivered to mitigate the CVHR. In one approach, the therapy is delivered by an implantable device. In another approach, the therapy is delivered by a patient-external device.

In one embodiment, the therapy may be a drug therapy, and may involve use of an implantable or patient-external drug delivery device. In another embodiment, the therapy is a cardiac pacing therapy. The cardiac pacing therapy may be delivered by an implantable device or a patient-external device.

According to one approach, delivering a cardiac pacing therapy involves adjusting one or more pacing parameters to mitigate the CVHR. In another approach, delivering the cardiac pacing therapy involves adjusting at least one of a pacing mode, a pacing rate, and a pacing pattern to mitigate the CVHR. Methods of the present invention may involve determining that the therapy mitigates the CVHR, and continuing the therapy or administering another therapy until the sleep disordered breathing is mitigated.

In accordance with another embodiment, a system includes a housing configured for implantation in a patient. The system further includes a sensor system, comprising a first sensor arrangement configured to sense one or more signals indicative of sleep disordered breathing, and a second sensor arrangement configured to sense a physiological signal that exhibits cyclical behavior due to repeated occurrences of sleep disordered breathing. A processor is provided in the housing and coupled to the sensor system. The processor is configured to detect repeated occurrences of sleep disordered breathing using the one or more signals sensed by the first sensor arrangement, and to detect changes of the physiological signal indicative of cyclic variation of heart rate.

The first sensor arrangement may includes a transthoracic impedance sensor. The second sensor arrangement may include at least one of an ECG sensor, blood pressure sensor, neural activity sensor, autonomic activity sensor, EMG sensor, EEG sensor, EOG sensor, photoplethysmography sensor, blood gas saturation sensor, intracardiac pressure sensor, and left ventricular transmural sensor.

According to a further embodiment, a system includes a housing configured for implantation in a patient. A pulse generator is provided in the housing and coupled to a cardiac lead system. A sensor system includes a first sensor arrangement configured to sense one or more signals indicative of sleep disordered breathing, and a second sensor arrangement configured to sense a physiological signal that exhibits cyclical behavior due to repeated occurrences of sleep disordered breathing. A processor is provided in the housing and coupled to the sensor system and the pulse generator. The processor is configured to detect repeated occurrences of sleep disordered breathing using the one or more signals sensed by the first sensor arrangement, detect changes of the physiological signal indicative of cyclic variation of heart rate, and deliver a cardiac electrical therapy to mitigate the CVHR.

One or both of the first and second sensor arrangements may be supported by the cardiac lead system. The processor may be configured to adjust one or more pacing parameters of the cardiac electrical therapy to mitigate the CVHR. The processor may be configured to adjust at least one of a pacing mode, a pacing rate, and a pacing pattern to mitigate the CVHR. The processor may be further configured to deliver or adjust a therapy to mitigate the sleep disordered breathing.

### BRIEF DESCRIPTION OF THE DRAWINGS

FIG. 1 shows various physiological signals that are adversely modulated as a result of repeated episodes of sleep disordered breathing;

FIG. 2 illustrates various processes associated with cyclic variation of heart rate detection in accordance with an embodiment of the present invention;

FIG. 3 illustrates a variety of physiological parameters or signals that may be monitored and analyzed to detect the presence of cyclic variation of heart rate;

FIG. 4A is an illustration of a heart rate waveform that exhibits oscillatory behavior relative to a baseline heart rate indicative of cyclic variation of heart rate;

FIG. 4B shows heart rate intervals (RR intervals) during normal breathing, and, more particularly, indicates respiratory sinus arrhythmia;

FIG. 4C is a plot of the frequency spectrum (i.e., power spectral density or PSD) of the heart rate signal shown in FIG. 4B, which can be used as a surrogate of respiration (e.g., abdominal respiration);

FIG. 4D shows a heart rate signal of a senior adult that experiences CVHR;

FIG. 4E shows a PSD estimation of the heart rate waveform shown in FIG. 4D;

FIGS. 4F-4I show Poincare plots developed from RR intervals and plots resulting from computation of Pearson's correlation coefficient;

FIG. 5 illustrates various processes associated with CVHR detection and treatment in accordance with an embodiment of the present invention;

FIG. 6 illustrates various processes associated with CVHR treatment in accordance with another embodiment of the present invention;

FIGS. 7A-7C are tachograms that illustrate the effectiveness of therapy that treats CVHR according to the principles of the present invention;

FIGS. 8-11 show several respiration waveforms that may be developed by a medical device implementing a sleep disorder detection methodology of the present invention;

FIG. 12 is a block diagram of a diagnostic and therapy delivery system according to an embodiment of the present invention; and

FIG. 13 is an illustration of a cardiac rhythm management system configured to implement sleep disorder diagnostics and CVHR detection and treatment in accordance with an embodiment of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which are shown by way of illustration, various embodiments by which the invention may be practiced. It is to be understood that other embodiments may be utilized, and structural and functional changes may be made without departing from the scope of the present invention.

An adequate quality and quantity of sleep is required to maintain physiological homeostasis. Prolonged sleep deprivation or periods of highly fragmented sleep ultimately will have serious health consequences. Chronic fragmented sleep may be associated with various cardiac or respiratory disorders affecting a patient's health and quality of life.

By way of example, a significant percentage of patients between 30 and 60 years experience some symptoms of disordered breathing, primarily during periods of sleep. Sleep disordered breathing is associated with excessive daytime sleepiness, systemic hypertension, increased risk of stroke, angina and myocardial infarction. Disturbed respiration can be particularly serious for patients concurrently suffering from cardiovascular deficiencies. Disordered breathing is particularly prevalent among congestive heart failure patients, and may contribute to the progression of heart failure.

It is estimated that about 50% of heart failure patients have such co-morbidity, especially central sleep apnea (CSA) and Cheyne-Stokes respiration (CSR). The actual mechanism between cardiovascular disease (CVD) or heart failure (HF) and sleep disordered breathing (SDB) is presently unclear. It is well accepted, however, that in the pathological loop between CVD/HF and SDB, cardiovascular disease or heart failure could result in periodic SDB, while periodic SDB results in acute changes in blood pressure and autonomic output, which can adversely worsen cardiovascular disease or heart failure status.

Cyclic variation of heart rate is a brady-tachycardia heart rate oscillation that can occur during repetitive sleep disordered breathing episodes. CVHR may be viewed as a pattern of sinus arrhythmia, characterized by progressive bradycardia at the onset of sleep disordered breathing (e.g., apnea, including central, obstructive, or combined central and obstructive) followed by abrupt tachycardia upon resumption of breathing. CVHR is associated with acute blood pressure surges, peripheral vasoconstriction, autonomic disbalances, and reduced cardiac output.

CVHR may have other adverse manifestations, in addition to those specified above. For example, CVHR is associated with recurrent hypoxia/re-oxygenation resulting from repeated episodes of apnea. CVHR may associate with shear stress changes induced by heart rate and blood pressure variability. CVHR may trigger endothelin (a potent vasoconstrictor) dysfunction. CVHR has been studied and reported as a signature of severe apnea in patients with normal sinus function and intact autonomic nervous system (ANS). CVHR has also been observed in patients with sick sinus node syndrome.

In the pathological loop between CVD/HF and SDB, it is believed that CVHR is a consistent cardiac manifestation indicating the acute link between cardiovascular disease/heart failure and sleep disordered breathing. It is further believed that the pathological loop between CVD/HF and SDB can be severed by controlling, mitigating, and/or eliminating CVHR.

The present invention is directed to methods and systems for detecting a sleep disorder or other disorder that, when occurring repeatedly, results in periodic or oscillatory changes in a physiologic parameter indicative of cyclic variation of heart rate. According to embodiments of the present invention, methods and systems of the present invention provide for detection of repeated episodes of sleep disordered breathing. After a series of sleep disordered breathing episodes has been detected, one or more parameters that exhibit periodic, cyclical, or oscillatory behavior due to the repeated sleep disordered breathing episodes are detected as indicators of CVHR.

According to other embodiments, therapy is delivered to mitigate detected CVHR. One or more of a variety of physiologic parameters may be monitored relative to therapeutic thresholds, and therapy may be adjusted or titrated to control, mitigate and/or eliminate CVHR. In this regard, CVHR is used as an abnormal hemodynamic indicator, and used to initiate or titrate therapy to stabilize or optimize cardiac output and mitigate blood pressure surges and other manifestations of CVHR. Therapy may also be delivered to control, mitigate and/or eliminate the patient's sleep disordered breathing, such as apnea (central, obstructive, or combined central and obstructive).

The following discussion is generally directed to embodiments of the invention that provide for detection of breathing disorders that are associated with occurrences of CVHR, and detection of indicators of CVHR after such breathing disorders are detected. Embodiments of the present invention are directed, in particular, to detection of sleep disordered breathing, including apnea (of any type) and hypopnea respiratory disorders. It is understood that embodiments of the present invention may be implemented in methods and systems that provide for detection of other forms of disordered breathing and is sleep disorders that may be associated with CVHR.

For example, involuntary movement disorders, such as periodic leg movement disorder (PLMD), may complicate or confound detection of CVHR. It may be desirable to detect PLMD or other involuntary movement disorder (e.g., restless leg syndrome), for example, as part of CVHR detection and/or verification, such as by detecting patient activity (e.g., by use of an EMG sensor) to discriminate apnea induced CVHR from other possible causes of CVHR.

Accordingly, the embodiments described below are provided for illustrative purposes, and are not to be regarded as limiting the scope of the present invention.

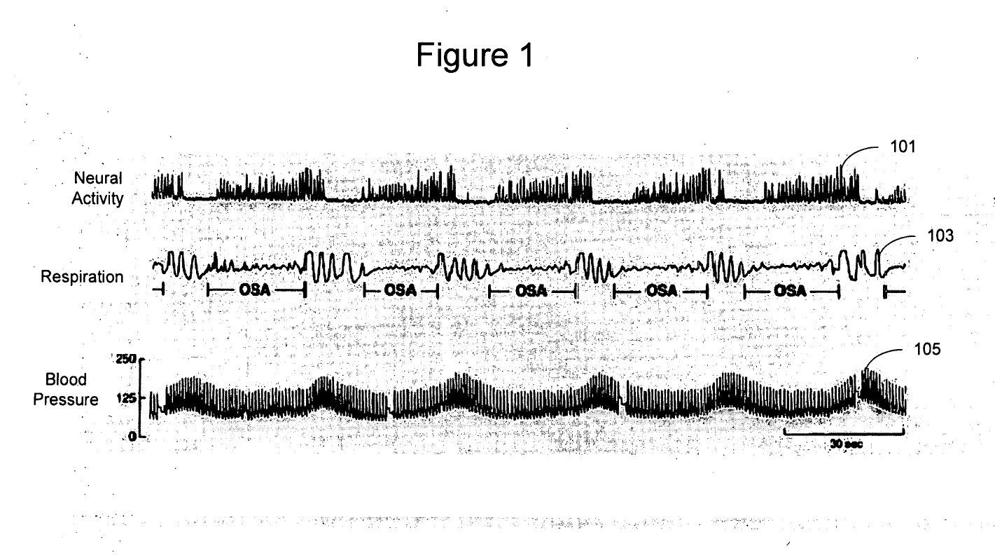

Turning now to the figures, FIG. 1 shows various physiological signals that are adversely modulated as a result of repeated episodes of sleep disordered breathing 103. In particular, it can be seen in FIG. 1 that incessant oscillations in sympathetic neural activity 101 and blood pressure 105 occur in concert with respiratory disturbances associated with obstructive sleep apnea 103. Neural activity and blood pressure represent two physiological signals that may be monitored for oscillatory behavior in the presence repeated apnea episodes, and are useful indicators for detecting the presence of CVHR. It is understood that physiological signals other than neural activity and blood pressure may be useful indicators for detecting the presence of CVHR, and include those physiological signals that exhibit periodic, cyclical, or oscillatory behavior due to the repeated sleep disordered breathing episodes.

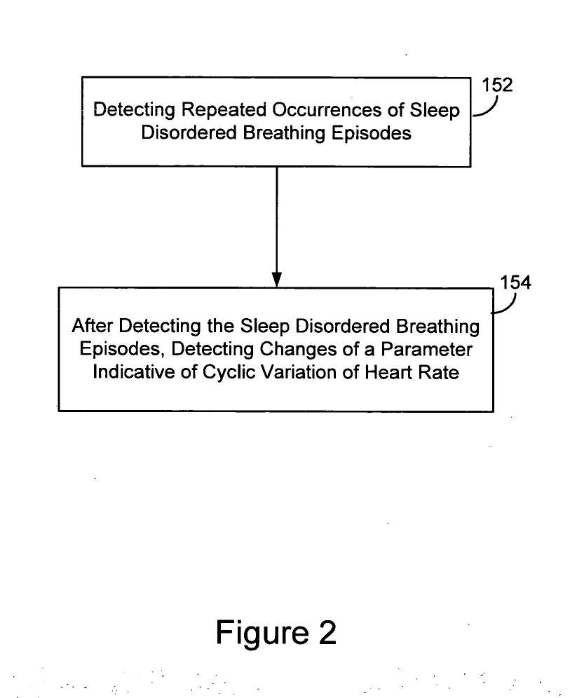

FIG. 2 illustrates various processes associated with CVHR detection in accordance with an embodiment of the present invention. According to FIG. 2, repeated occurrences of sleep disordered breathing are detected 152, such as by use of an implantable device. After detecting the repeated occurrences of sleep disordered breathing, such as a series of SDB episodes, the method involves detecting 154 changes of a parameter indicative of cyclic variation of heart rate, such as by use of an implantable device. It is understood that the methodology illustrated in FIG. 2 may be performed using a patient-external device or a combination of patient-internal and patient-external devices.

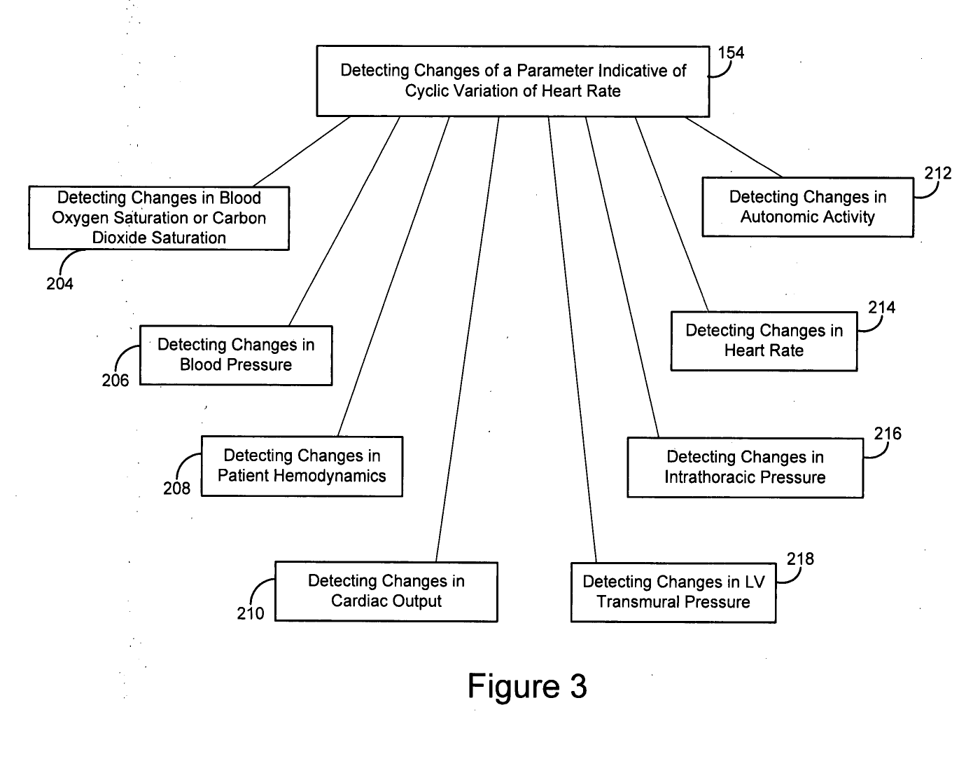

FIG. 3 illustrates a variety of physiological parameters or signals that may be monitored and analyzed to detect the presence of CVHR. As was previously stated, a variety of physiological parameters or signals may be useful for detecting the presence of CVHR. In general, useful CVHR detection are those that exhibit periodic, cyclical, or oscillatory behavior due to the repeated sleep disordered breathing episodes. Heart rate 214 (e.g., via electrocardiograms) represents a parameter that can be monitored and used as a direct indicator of CVHR. Other physiological parameters or signals may be useful surrogate indicators of CVHR. Such surrogate indicators may be used to detect the presence of CVHR, or may be used to confirm/verify the presence of CVHR as detected by use of a different parameter or signal, such as heart rate.

As is shown in FIG. 3, changes in blood gas saturation 204 during repeated sleep disordered breathing episodes may be a useful indicator of the presence of CVHR. One or both of blood oxygen saturation and blood carbon dioxide saturation may be useful blood gas saturation parameters. Changes in blood pressure 206 and autonomic activity 212 represent useful parameters for detecting the presence of CVHR, as was discussed previously.

Any of a number of hemodynamic parameters 208 that exhibit periodic, cyclical, or oscillatory behavior due to the repeated sleep disordered breathing episodes may be monitored and analyzed to detect the presence of CVHR. For example, changes in cardiac output 210 may be a particularly useful monitoring parameter for CVHR detection. Changes in intrathoracic pressure 216 or left ventricular transmural pressure 218 may be monitored and analyzed to detect the presence of CVHR.

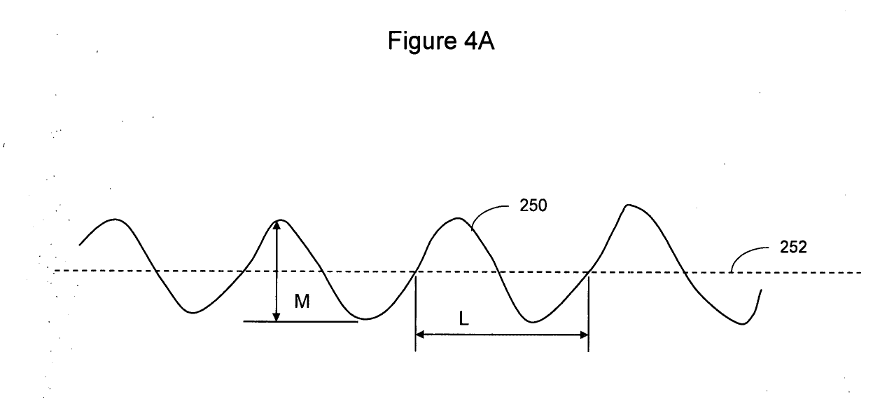

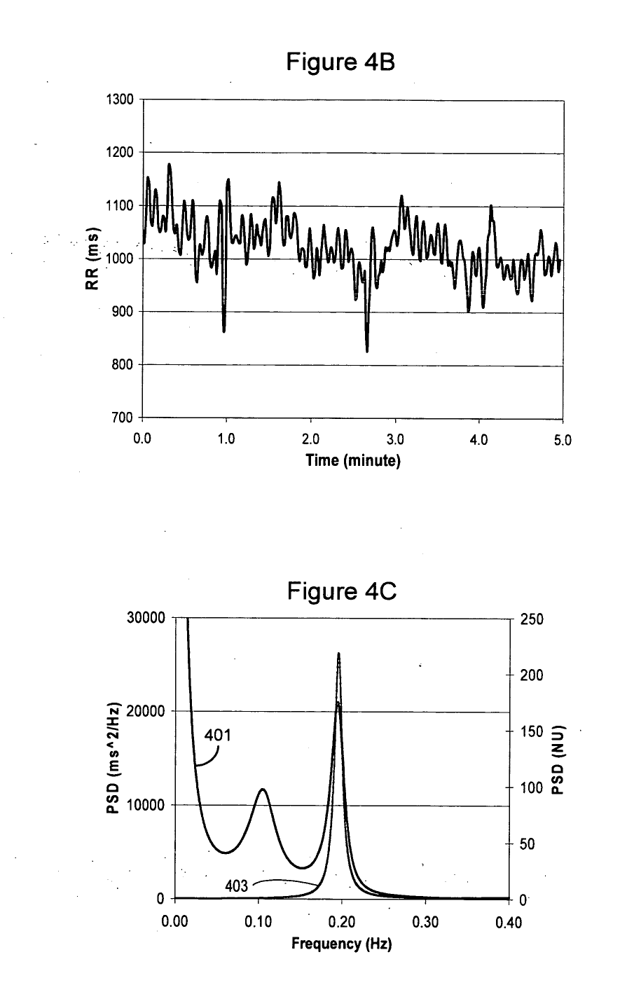

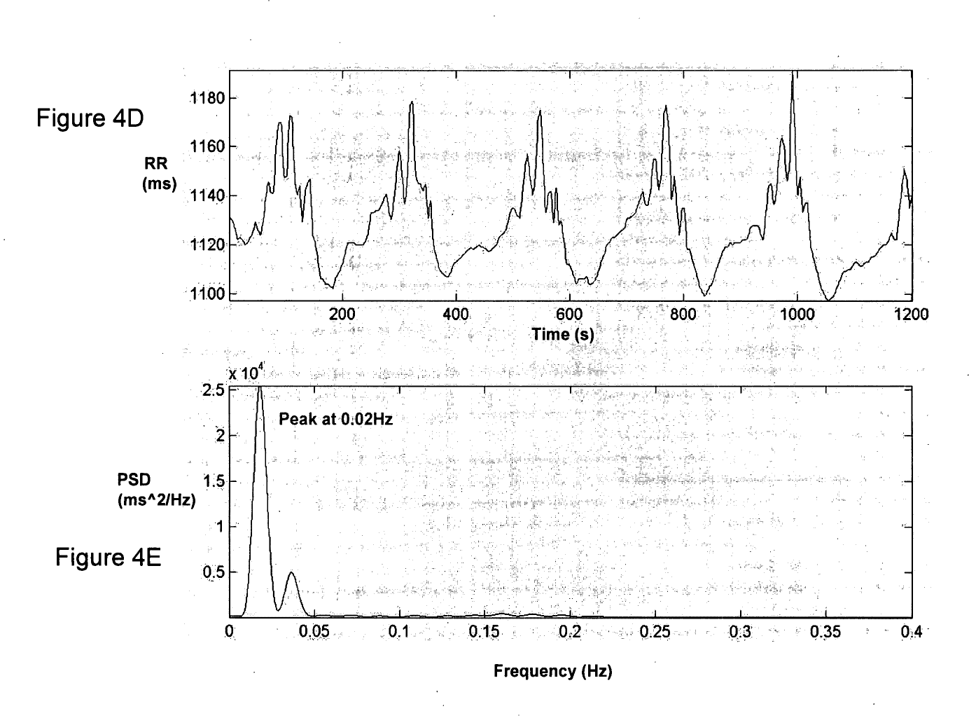

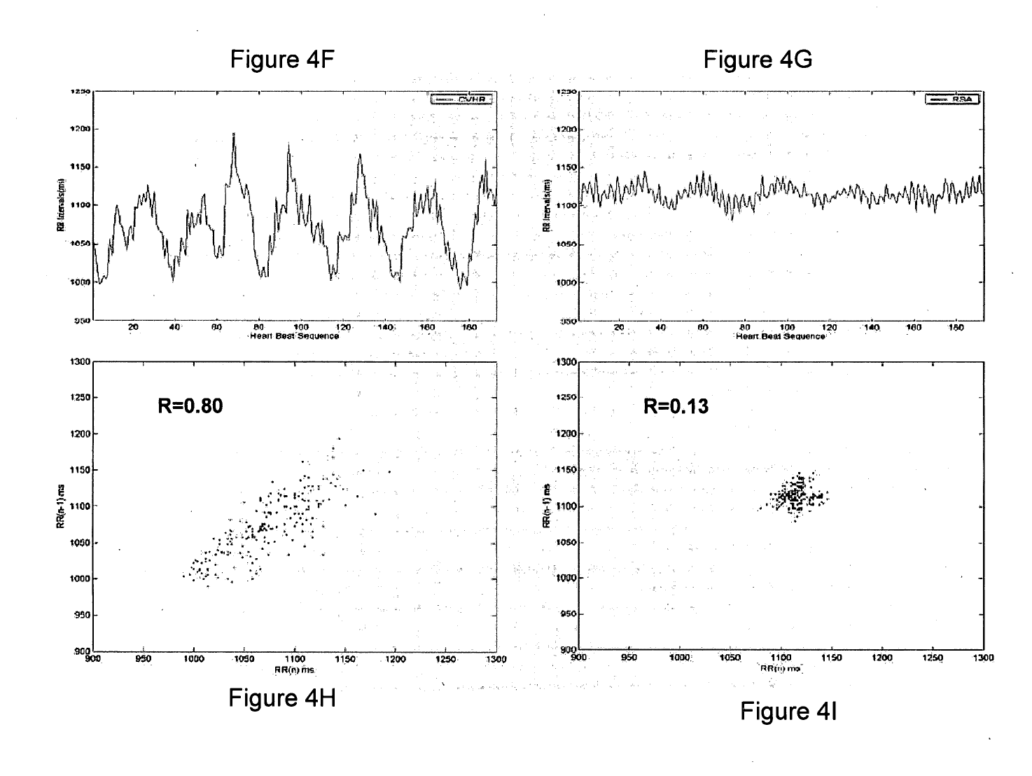

FIG. 4A is an illustration of a heart rate waveform 250 that exhibits oscillatory behavior relative to a baseline heart rate 252. The cyclic perturbations of waveform 250 shown in FIG. 4A are consistent with CVHR that occurs in the presence of repeated sleep disordered breathing episodes. Waveform 250 may be analyzed using a variety of techniques to detect the presence of oscillation and, in various embodiments, stability of such oscillations. Detection of the stability or persistence in the cyclic behavior of waveform 250 may be particularly useful for facilitating automatic and/or manual therapy decisions for treatment of CVHR. Such therapy decisions may include selection of an appropriate therapy, adjustment of a particular therapy presently being delivered, titration of a given therapy to mitigate CVHR and/or stabilize a hemodynamic indicator of CVHR (e.g., blood pressure, neural activity, cardiac output), and/or to terminate a given therapy.

A waveform indicative of CVHR (e.g., heart rate waveform 250 or other physiological waveform) may be analyzed in terms of one or more waveform features in order to detect the presence and/or stability of waveform oscillation indicative of CVHR. FIG. 4A shows two such useful features of waveform 250, amplitude (A) and cycle length (L).

According to another approach, cycle length (L), peak-to-peak heart rate amplitude (A), periodicity, and stability of periodicity may be used to detect/verify CVHR. For example, the following equations may be used:
t₁<L<t₂ [1]
HR_{MAX}−HR_{MIN} within L>T_{threshold} [2]
Periodicity>n consecutive cycles [3]
satisfying both Eq. [1] and Eq. [2]
   Stability of Periodicity over n consecutive cycles [4]
   where t₁ is about 25 seconds, t₂ is about 100 seconds (noting that, normally, CVHR frequency can range from about 0.01 Hz to about 0.1 Hz), T_{threshold} is about 5 bpm, n is at least 3, and stability of the periodicity may be represented by the standard deviation of L (over n consecutive cycles, for example). Tachograms may be developed based on heart rate, from which CVHR may be detected in the above-described manner. Visual inspection of such tachograms can also form the basis for CVHR detection, such as by a clinician. Conclusions reached by a clinician after visual inspection of a patient's tachogram (or other CVHR diagnostic output, such a frequency or Poincare analysis as described herein) may be used to confirm CVHR and select or adjust a therapy for treating CVHR.

The frequency of waveform oscillation may also be readily computed. The frequency of waveform oscillation is considered to be a robust waveform feature that may used to detect CVHR. Amplitude (i.e., peak-to-peak amplitude) in combination with frequency may further enhance CVHR detection. For example, frequency or periodicity of waveform 250 may be used to detect the presence of CVHR, and measured amplitude of the cyclic waveform 250 may be used to make therapy delivery or adjustment decisions, such as cardiac pacing rates, modes, and/or patterns.

FIG. 4B shows a heart rate signal (RR intervals) plotted as a function of time (minutes) during normal patient breathing. FIG. 4B indicates a patient's respiratory sinus arrhythmia. FIG. 4C is a plot of the frequency spectrum (PSD) of the heart rate signal shown in FIG. 4B, which can be used as a surrogate of respiration (e.g., abdominal respiration). In particular, FIG. 4C is a plot of the PSD estimation 401 of the heart rate signal shown in FIG. 4B, and the PSD estimation 403 of the patient's abdominal respiration. It can be seen from FIG. 4C that the peaks of PSD estimations 401 and 403 are in alignment at a frequency of about 0.20 Hz.

FIG. 4D shows a heart rate signal of a senior adult that experiences CVHR. The cyclical behavior of heart rate indicative of CVHR resulting from repeated SDB episodes can readily be seen in FIG. 4D. FIG. 4E shows a PSD estimation of the heart rate waveform shown in FIG. 4D. It can be seen in FIG. 4D that the peak of the PSD estimation resides at a frequency of about 0.02 Hz. It has been observed that a PSD estimation of a heart rate waveform developed from a child that experiences SDB and CVHR has a peak that resides at a frequency of about 0.045 Hz.

It is believed that frequencies associated with CVHR can range from about 0.01 Hz and about 0.1 Hz, and typically range between about 0.01 Hz and about 0.05 Hz. In one illustrative example, CVHR may be detected using the generalized equation 0.01 Hz<f_{CVHR}<0.1 Hz. In another illustrative example, CVHR may be detected using the generalized equation 0.01 Hz<f_{CVHR}<0.05 Hz. The presence of CVHR may be confirmed by satisfaction of a selected one of the above equations over n CVHR cycles, where n is about 5. Other useful frequency-based techniques for detecting CVHR include those involving Fourier, Welch, and auto-regressive moving average (ARMA) analysis.

As is indicated in Equation [4] above, it may be useful to determine the stability of detected CVHR. One approach to determining CVHR stability involves determining deviations in the frequency of heart rate (or other parameter) oscillation over n samples, where n is at least 3. For example, CVHR stability may be determined where the frequency of waveform oscillation over n samples deviates by no more that about 5-10%. In terms of time, a typical CVHR cycle (e.g., apnea onset-recovery cycle) may be about 50 seconds in duration. Stability of a series of such CVHR cycles may be confirmed if cycle-to-cycle deviations do not exceed +/−5 seconds. Various other techniques may be used to determine the stability of CVHR over n samples, include those involving computation of standard deviation.

Detection of CVHR may be implemented using other techniques. FIGS. 4F-4I show Poincare plots developed from RR intervals and plots resulting from computation of Pearson's correlation coefficient. CVHR detection may involve determining if the correlation coefficient exceeds a predetermined threshold or falls within a threshold range. Useful techniques for determining variability in heart rate using Poincare plots that can be employed in the context of CVHR detection according to the present invention are described in U.S. Pat. No. 6,731,974, which is hereby incorporated herein by reference. Useful techniques for determining variability in autonomic activity using Poincare plots that can be employed in the context of CVHR detection according to the present invention are described in U.S. Pat. No. 5,682,901, which is hereby incorporated herein by reference.

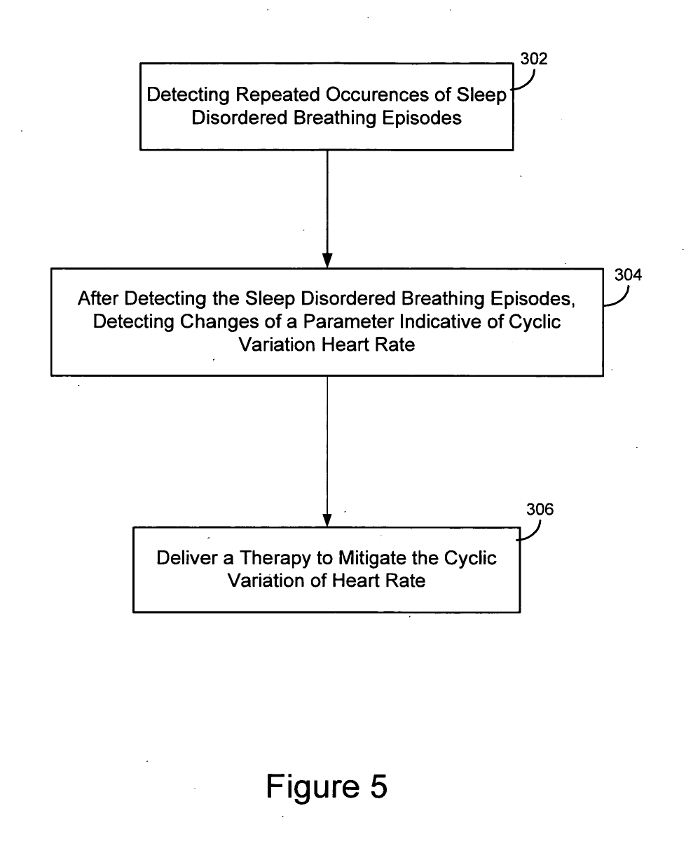

FIG. 5 illustrates various processes associated with CVHR detection and treatment in accordance with an embodiment of the present invention. According to FIG. 5, repeated occurrences of sleep disordered breathing are detected 302, such as by use of an implantable device. After detecting the repeated occurrences of sleep disordered breathing, such as a series of SDB episodes, the method involves detecting 304 changes of a parameter indicative of cyclic variation of heart rate, such as by use of an implantable device. A therapy is delivered 306 to treat the detected CVHR, such as by use of an implantable device. It is understood that the methodology illustrated in FIG. 5 may be performed using a patient-external device or a combination of patient-internal and patient-external devices.

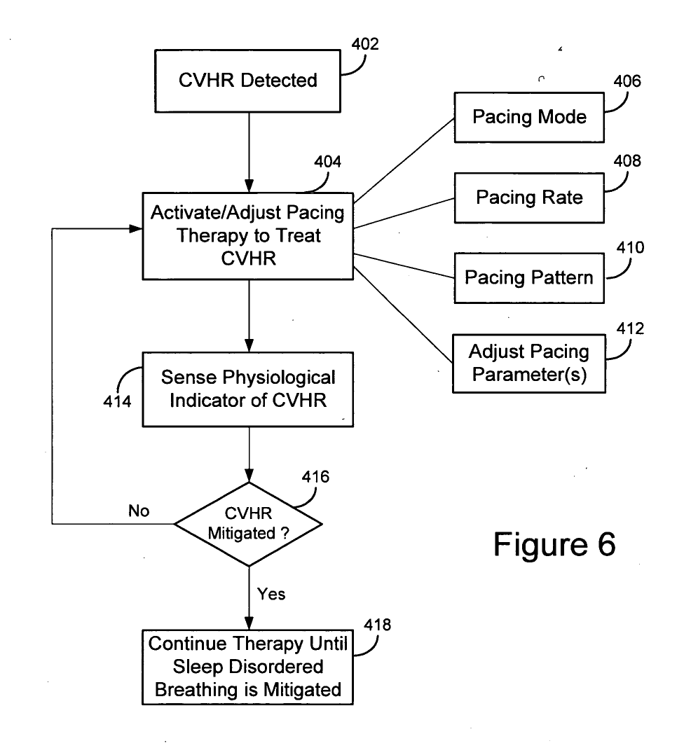

FIG. 6 illustrates various processes associated with CVHR treatment in accordance with an embodiment of the present invention. According to FIG. 6, CVHR is detected 402, such as by use of a technique described herein. After detecting CVHR 402, a cardiac pacing therapy is activated 404 to treat the CVHR. One or more physiological indicators of CVHR are monitored 414 in order to assess the efficacy of the pacing therapy. If the pacing therapy results in mitigation 416 of the CVHR, the therapy may be continued or titrated to further mitigate or eliminate the CVHR.

If CVHR is not mitigated 416, the pacing therapy may adjusted 404 or another pacing therapy may be selected (and further titrated according to FIG. 6). For example, treating CVHR may involve selection of one or several pacing modes 406. The pacing rate 408 and/or pacing pattern 410 may be selected or adjusted to treat the CVHR. One or more pacing parameters 412 may be adjusted to titrate therapy delivery. Such pacing parameters may include AV delay, V-V delay, selection of atrial overdrive pacing (AOP) in dual chamber applications, and AOP in cardiac resynchronization therapy (CRT) applications (e.g., VDD vs. DDD modes). It is noted that amplitude (A) of cyclic waveform indicative of CVHR may be used to effect pacing configuration (and parameter selection) decisions, such as minimum and maximum pacing rates.

As is further shown in FIG. 6, it may be desirable to continue delivery of a therapy 418 to mitigate the patient's sleep disordered breathing. The SDB therapy may be the same therapy that is used to treat CVHR, or may be different therapy that is targeted for SDB therapy. If different therapies are delivered for separately treating CVHR and SDB, these therapies may be delivered in serial fashion or in parallel (i.e., concurrently).

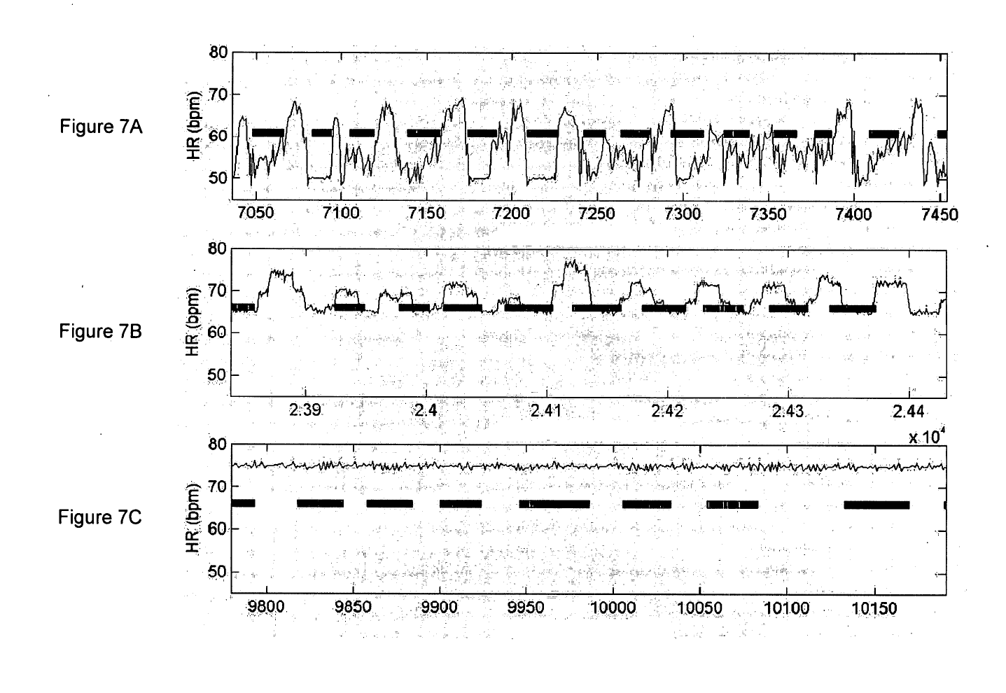

FIGS. 7A-7C are tachograms that illustrate the effectiveness of therapy that treats CVHR according to the principles of the present invention. The bars in FIGS. 7A-7C represent episodes of apnea. As can be seen in FIG. 7A, the patient's heart rate oscillates relative to a baseline heart rate of about 55 bpm due to repeated apnea episodes, evidencing the presence of CVHR. In response to detecting repeated apnea episodes and the presence of CVHR, cardiac electrical therapy (or other therapy) may be delivered to treat the CVHR, such as in a manner previously described.

FIG. 7B represents the patient's tachogram sometime after initiation of CVHR mitigation therapy. FIG. 7B evidences mitigation of CVHR due to the delivered therapy, as can be seen by the reduced amplitude of heart rate oscillations and increase of the patient's baseline heart rate. The therapy delivered to the patient may be titrated to further mitigate CVHR. FIG. 7C represents significant mitigation of CVHR, as can be seen by a further reduction in the amplitude of heart rate oscillations and further increase of the patient's baseline heart rate to about 75 bpm.

It is understood that various therapies other than cardiac electrical therapy may be employed to treat the CVHR in accordance with the methodology illustrated in FIG. 6. For example, a drug therapy may be used (alone or in combination with a pacing therapy or other therapy) to treat a patient's CVHR consistent with the approach illustrated in FIG. 6. One such drug therapy may involve controlled delivery of a drug that mitigates or eliminates CVHR, such as atropine. It has been observed that atropine eliminates CVHR, while propranolol, for example, had no effect. Such a drug therapy may be administered by a clinician intravenously, for example, or automatically via a drug delivery apparatus (e.g., drug pump).

Other therapies may be delivered and/or titrated in response to detecting CVHR. Such other therapies include muscle stimulation therapy, CPAP, ventilation, or other respiratory therapy, and vagal nerve stimulation therapy. These and other therapies may be delivered alone or in combination (e.g., serially or in parallel) to mitigate or eliminate CVHR. Such therapies may also be delivered and/or titrated to mitigate or eliminate a patient's disordered breathing.

Such therapies may be administered directly by a clinician or automatically via an appropriate therapy delivery apparatus (e.g., drug delivery device, pacemaker, muscle stimulator, nerve stimulator, CPAP or ventilator unit). Depending on the type of therapy involved, the therapy delivery apparatus may be a patient-external apparatus or an implantable apparatus. Control and/or titration of the therapy may be effected by a patient-internal or -external device, such as a cardiac rhythm management device, and may be facilitated or enhanced by use of an advanced patient management system or other patient-external system.

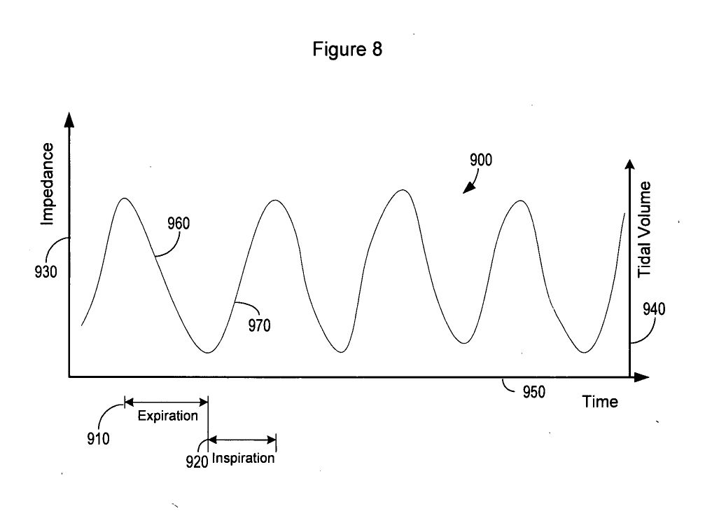

Referring now to FIGS. 8-11, several respiration waveforms are shown that may be developed by a medical device implementing a sleep disorder detection methodology of the present invention. Respiratory waveforms of the type shown in FIGS. 8-11 may be developed using a transthoracic impedance sensor of an implantable medical device, such as a cardiac monitor or cardiac rhythm management (CRM) device. Suitable cardiac monitors and CRM devices include endocardial, transvenous, and subcutaneous, non-intrathoracic devices.

The respiratory waveforms shown in FIGS. 8-11 represent one of several types of physiological signals that may be used to sense or detect a sleep disorder associated with CVHR. In these illustrative examples, the respiratory waveforms are analyzed, typically by implantable device circuitry, for purposes of detecting disordered breathing, such as apnea and hypopnea. Aberrations in the respiratory waveforms are analyzed to detect apnea and hypopnea, such as on a per-hour basis so that an apnea-hypopnea index (AHI) may be computed.

With reference to FIG. 8, an impedance signal 900 is illustrated, which is useful for determining sleep, sleep state, and sleep disordered breathing. The impedance signal 900 may be developed, for example, from an impedance sense electrode in combination with a cardiac monitor or CRM device, for example. The impedance signal 900 is proportional to the transthoracic impedance, illustrated as an impedance 930 on the abscissa of the left side of the graph in FIG. 8.

The impedance 930 increases 970 during any respiratory inspiration 920 and decreases 960 during any respiratory expiration 910. The impedance signal 900 is also proportional to the amount of air inhaled, denoted by a tidal volume 940, illustrated on the abscissa of the right side of the graph in FIG. 8. The variations in impedance during respiration, identifiable as the peak-to-peak variation of the impedance signal 900, may be used to determine the respiration tidal volume 940. Tidal volume 940 corresponds to the volume of air moved in a breath, one cycle of expiration 910 and inspiration 920. A minute ventilation may also be determined, corresponding to the amount of air moved per a minute of time 950 illustrated on the ordinate of the graph in FIG. 8.

Breathing disorders may be determined using the impedance signal 930. During non-REM sleep, a normal respiration pattern includes regular, rhythmic inspiration—expiration cycles without substantial interruptions. When the tidal volume of the patient's respiration, as indicated by the transthoracic impedance signal, falls below a hypopnea threshold, then a hypopnea event is declared. For example, a hypopnea event may be declared if the patient's tidal volume falls below about 50% of a recent average tidal volume or other baseline tidal volume value. If the patient's tidal volume falls further to an apnea threshold, e.g., about 10% of the recent average tidal volume or other baseline value, an apnea event is declared.

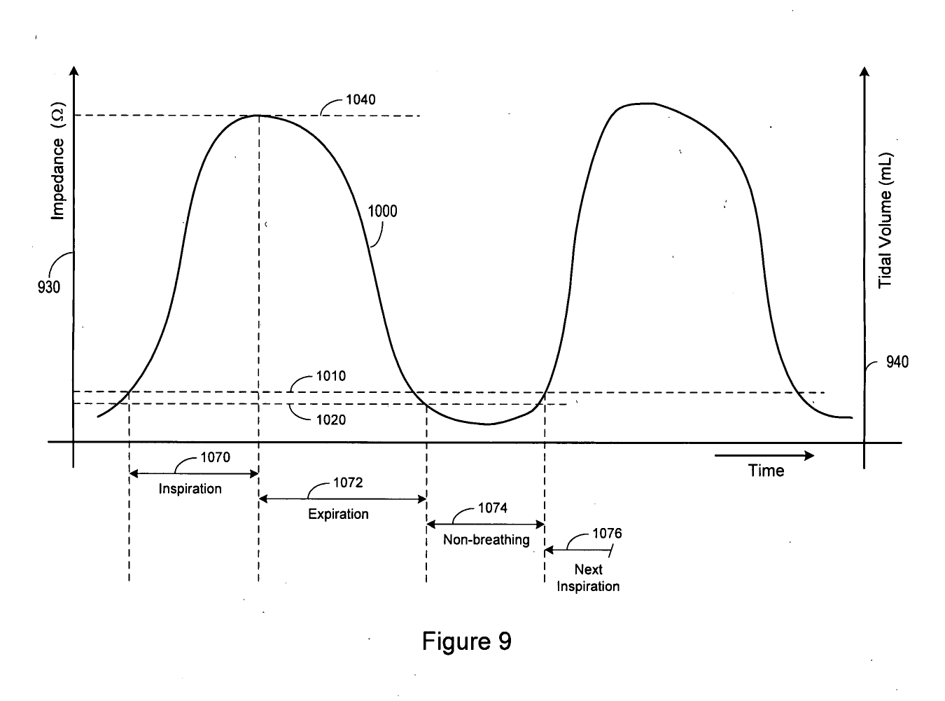

FIGS. 9-11 are graphs of transthoracic impedance and tidal volume, similar to FIG. 8 previously described. As in FIG. 8, FIGS. 9-11 illustrate the impedance signal 1000, 1100, 1200 proportional to the transthoracic impedance, again illustrated as impedance 930 on the abscissa of the left side of the graphs in FIGS. 9-11. The impedance 930 increases during any respiratory inspiration and decreases during any respiratory expiration. As before, the impedance signal 1000, 1100, 1200 is also proportional to the amount of air inhaled, denoted the tidal volume 940, illustrated on the abscissa of the right side of the graph in FIGS. 9-11. The magnitude of variations in impedance and tidal volume during respiration are identifiable as the peak-to-peak variation of the impedance signal 1000, 1100, 1200.

FIG. 9 illustrates respiration intervals useful for detecting disordered breathing in accordance with embodiments of the invention. Detection of disordered breathing may involve defining and examining a number of respiratory cycle intervals. A respiration cycle is divided into an inspiration period corresponding to the patient inhaling, an expiration period, corresponding to the patient exhaling, and a non-breathing period occurring between inhaling and exhaling.

Respiration intervals are established using an inspiration threshold 1010 and an expiration threshold 1020. The inspiration threshold 1010 marks the beginning of an inspiration period 1070 and is determined by the transthoracic impedance signal 1000 rising above the inspiration threshold 1010. The inspiration period 1070 ends when the transthoracic impedance signal 1000 is a maximum 1040. The maximum transthoracic impedance signal 1040 corresponds to both the end of the inspiration interval 1070 and the beginning of an expiration interval 1072. The expiration interval 1072 continues until the transthoracic impedance 1000 falls below an expiration threshold 1020. A non-breathing interval 1074 starts from the end of the expiration period 1072 and continues until the beginning of a next inspiration period 1076.

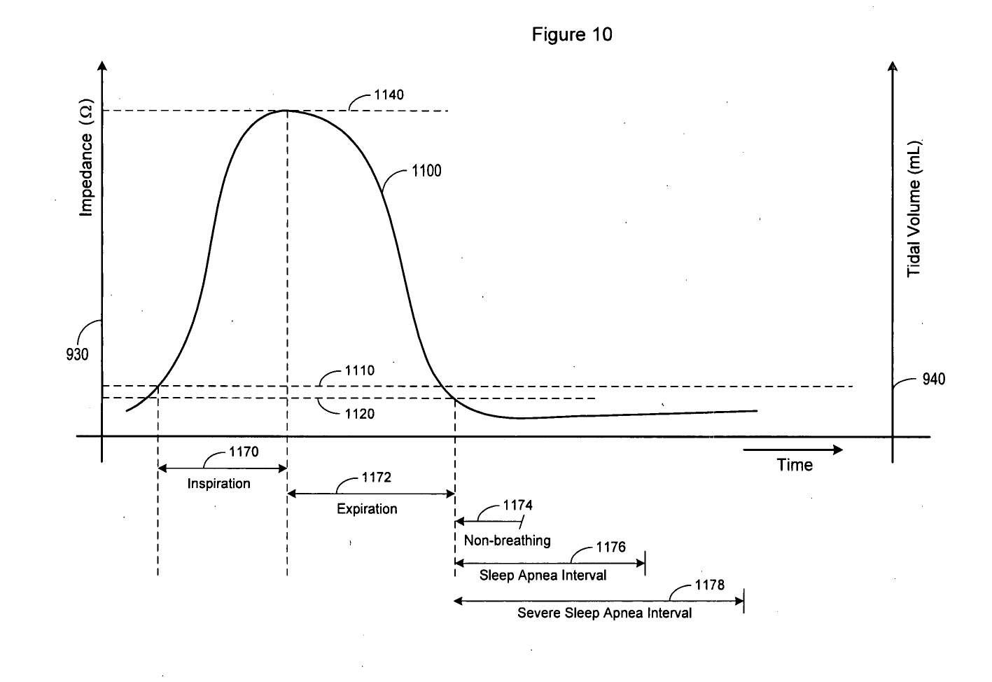

Detection of sleep disordered breathing events such as sleep apnea and severe sleep apnea is illustrated in FIG. 10. The patient's respiration signals are monitored and the respiration cycles are defined according to an inspiration 1170, an expiration 1172, and a non-breathing 1174 interval as described in connection with FIG. 9. A condition of sleep apnea is detected when a non-breathing period 1174 exceeds a first predetermined interval 1176, denoted the sleep apnea interval. A condition of severe sleep apnea is detected when the non-breathing period 1174 exceeds a second predetermined interval 1178, denoted the severe sleep apnea interval. For example, sleep apnea may be detected when the non-breathing interval exceeds about 10 seconds, and severe sleep apnea may be detected when the non-breathing interval exceeds about 20 seconds.

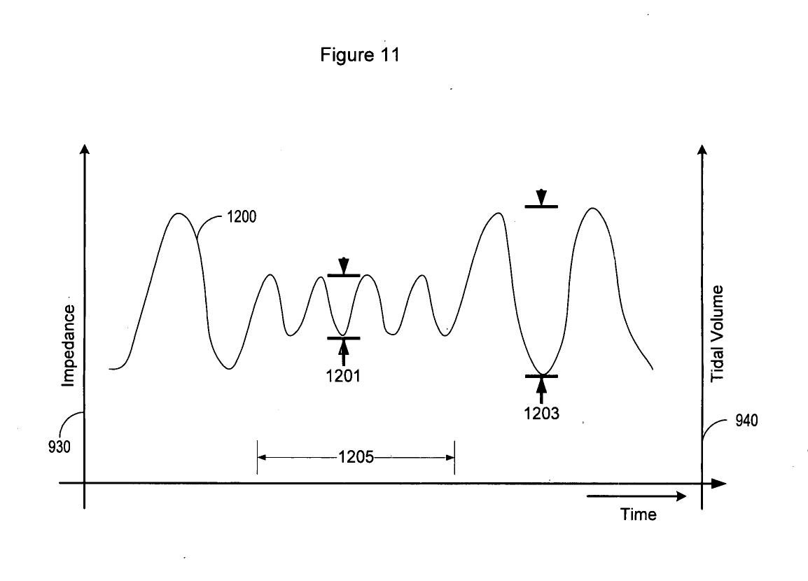

Hypopnea is a condition of sleep disordered breathing characterized by abnormally shallow breathing. FIG. 11 is a graph of tidal volume derived from transthoracic impedance measurements. The graph of FIG. 11 illustrating the tidal volume of a hypopnea episode may be compared to the tidal volume of a normal breathing cycle illustrated previously in FIG. 8, which illustrated normal respiration tidal volume and rate. As shown in FIG. 11, hypopnea involves a period of abnormally shallow respiration, possible at an increased respiration rate.

Hypopnea is detected by comparing a patient's respiratory tidal volume 1203 to a hypopnea tidal volume 1201. The tidal volume for each respiration cycle may be derived from transthoracic impedance measurements acquired in the manner described previously. The hypopnea tidal volume threshold may be established by, for example, using clinical results providing a representative tidal volume and duration of hypopnea events. In one configuration, hypopnea is detected when an average of the patient's respiratory tidal volume taken over a selected time interval falls below the hypopnea tidal volume threshold. Furthermore, various combinations of hypopnea cycles, breath intervals, and non-breathing intervals may be used to detect hypopnea, where the non-breathing intervals are determined as described above.

In FIG. 11, a hypopnea episode 1205 is identified when the average tidal volume is significantly below the normal tidal volume. In the example illustrated in FIG. 11, the normal tidal volume during the breathing process is identified as the peak-to peak value identified as the respiratory tidal volume 1203. The hypopnea tidal volume during the hypopnea episode 1205 is identified as hypopnea tidal volume 1201. For example, the hypopnea tidal volume 1201 may be about 50% of the respiratory tidal volume 1203. The value 50% is used by way of example only, and determination of thresholds for hypopnea events may be determined as any value appropriate for a given patient. In the example above, if the tidal volume falls below 50% of the respiratory tidal volume 1203, the breathing episode may be identified as a hypopnea event, originating the measurement of the hypopnea episode 1205.

Sleep disorder detection according to the present invention may employ a wide variety of sensors, implantable and non-implantable medical devices, systems and interfaces for acquiring manually-reported patient data, sleep disorder detection techniques, and therapies to treat sleep disorders. The embodiments discussed herein represent several non-limiting illustrative implementations.

These and other implementations for detecting conditions associated with sleep disorders may also provide for detection that a patient is asleep. A method of sleep detection is described in commonly owned U.S. patent application Ser. No. 10/309,771, filed Dec. 4, 2002, which is incorporated herein by reference in its entirety. In addition, classification of sleep state, including classification of rapid eye movement sleep (REM sleep) and non-REM sleep may also be used to enhance sleep detection and/or to determine the duration of various sleep states. Sensing abnormal sleep state durations may be indicative of restless sleep due to sleep apnea for example. Methods and systems involving classifying the patient's sleep state are described in commonly owned U.S. patent application Ser. No. 10/643,006, filed Aug. 18, 2003 under Attorney Docket No. GUID.060PA, which is hereby incorporated herein by reference.

Detection of a sleep disorder may involve detecting one or more conditions indicative of sleep disordered breathing (SDB). Methods and systems for detection and treatment of disordered breathing is described in commonly owned U.S. patent application Ser. No. 10/643,203, filed Aug. 18, 2003 under Attorney Docket No. GUID.059PA, which is hereby incorporated herein by reference. Another implementation of SDB detection includes detection and analysis of respiratory waveform patterns. Methods and systems for detecting disordered breathing based on respiration patterns are more fully described in commonly owned U.S. patent application Ser. No. 10/309,770, filed Dec. 4, 2002 under Attorney Docket No. GUID.054PA and U.S. patent application Ser. No. 10/309,771, filed Dec. 4, 2002 under Attorney Docket No. GUID.064PA, which are hereby incorporated herein by reference. Methods and systems for detecting disordered breathing based on ECG signal analysis are more fully described in U.S. Publication No. 2003/0055348, which is hereby incorporated herein by reference.

Detection of a sleep disorder may also involve detecting one or more conditions relating to involuntary muscle movement disorders, such as restless leg syndrome, periodic limb movement disorder, and bruxism, for example. Systems and techniques for detecting involuntary muscle movement disorders that may be implemented in accordance with the present invention are disclose in commonly owned U.S. patent application Ser. No. 10/920,675, filed Aug. 17, 2004 under GUID.106PA; Ser. No. 10/939,834, filed Sep. 13, 2004 under Attorney Docket No. GUID.127PA; and Ser. No. 10/939,639, filed Sep. 13, 2004 under Attorney Docket No. GIUD.141PA, all of which are hereby incorporated herein by reference.

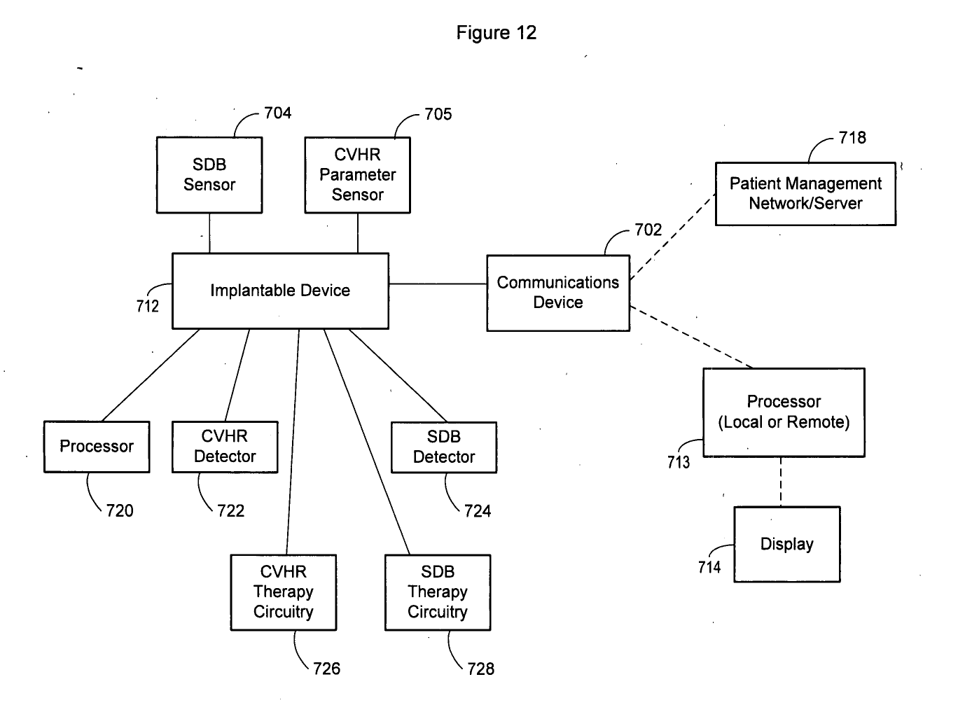

FIG. 12 is a block diagram of a diagnostic and therapy delivery system according to an embodiment of the present invention. According to the embodiment shown in FIG. 12, an implantable device 712 is configured for detecting sleep disorders and cyclic variation of heart rate. The implantable device 712 is further configured for delivering therapy to treat CVHR and sleep disorders. The implantable device 712 may be a cardiac rhythm management or monitoring system. The implantable device 712 may also be a nerve stimulation device or a drug therapy device. In other configurations, the device 712 need not be an implantable device. For example, the device 712 may be a respiration therapy device, such as a positive airway pressure device.

According to the embodiment shown in FIG. 12, the implantable device 712 includes a processor, such as a microprocessor or other logic-based processing device. One or more sleep disordered breathing sensors 704 are coupled to, or incorporated as part of, implantable device 712. The SDB sensors 704 are configured to sense a physiologic parameter useful in detecting the presence of sleep disordered breathing, such as those described herein.

An SDB detector 728, coupled to, or incorporated as part of, implantable device 712, is configured to detect the presence of sleep disordered breathing. The SDB detector 728 preferably implements a sleep disordered breathing detection methodology as described herein. SDB therapy circuitry 728 is coupled to, or incorporated as part of, implantable device 712. SDB therapy circuitry 728 is configured to deliver a therapy to treat detected sleep disordered breathing, such as the SDB therapies described herein.

One or more CVHR parameter sensors 705 are coupled to, or incorporated as part of, implantable device 712. The CVHR parameter sensors 705 are configured to sense a physiologic parameter useful in detecting CVHR, such as those described herein. A variety of physiological parameters that exhibit periodicity or oscillatory behavior due to repeated episodes of sleep disordered breathing may be sensed by CVHR parameter sensors 705.

A CVHR detector 726, coupled to, or incorporated as part of, implantable device 712, is configured to detect the presence of CVHR. The CVHR detector 726 preferably implements a methodology for detecting cyclic variation of heart rate as described herein. CVHR therapy circuitry 726 is coupled to, or incorporated as part of, implantable device 712. CVHR therapy circuitry 726 is configured to deliver a therapy to treat detected CVHR, such as by implementing the CVHR therapies described herein. Although described generally as being implantable, it is understood that all or some of the sensor(s) 704, 705, detectors 722, 724, and therapy circuitry 726, 728 may be implemented in patient-external devices or systems in various embodiments.

The sensors of FIG. 12 may include one or more of a transthoracic impedance sensor, minute ventilation sensor, EMG sensor, EEG sensor, electrocardiogram (ECG) sensor, neural activity sensor, accelerometer, posture sensor, proximity sensor, electrooculogram (EOG) sensor, photoplethysmography sensor, blood gas saturation sensor (e.g., oximetry sensor), blood pressure sensor, peripheral arterial tonography sensor, intracardiac pressure sensor, left ventricular transmural sensor, and/or other sensors useful in sensing conditions associated with CVHR and sleep disorders.

The device 712 may be configured to communicate with a patient-external system via communications device 702. The patient-external system may be a programmer, home/bed-side system, portable communicator, or interface to a patient management network/sever 718, such as an advanced patient management system. The patient-external system typically includes a processor 713 and is typically coupled to a display 714.

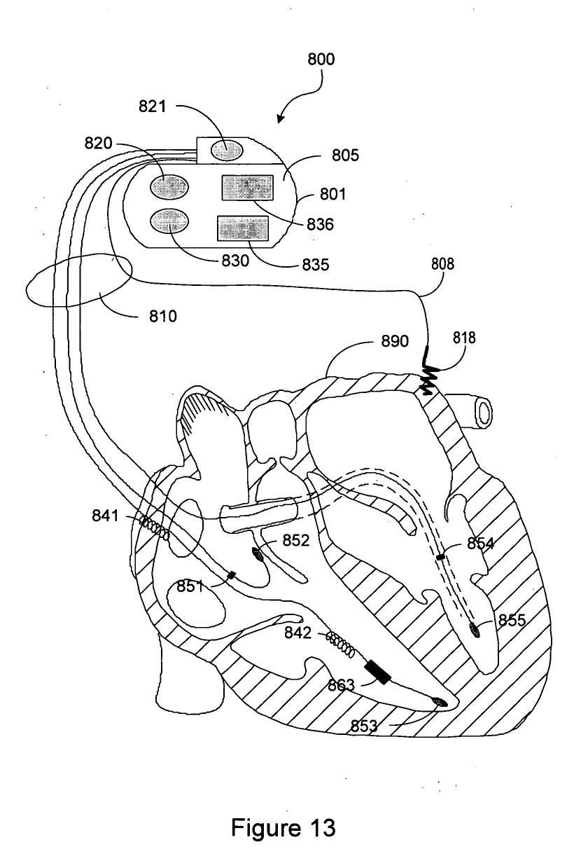

FIG. 13 is an illustration of a cardiac rhythm management system configured to implement sleep disorder diagnostics and CVHR detection and treatment in accordance with an embodiment of the present invention. The system 800 shown in FIG. 13 may be configured to include circuitry and functionality for sleep disorder and CVHR detection and treatment in accordance with embodiments of the invention. Although system 800 is described as a CRM device in FIG. 13, it is understood that sleep disorder diagnostics and CVHR detection and treatment circuitry and/or functionality may be implemented in a wide range of medical devices, including implantable, partially implantable, and non-implantable devices. Examples of these devices include monitoring, diagnostic, and/or therapeutic devices, such as cardiac monitoring and/or stimulation devices, respiration monitoring and therapy devices, implantable drug delivery devices, and neurostimulation devices, among others.

In the illustrative example shown in FIG. 13, CVHR detection circuitry 836 is configured as a component of a pulse generator 805 of CRM device 800. Sleep disorder diagnostic circuitry 835 is configured as a component of pulse generator 805. CVHR detection circuitry 836 and sleep disorder diagnostic circuitry 835 may be implemented in hardware, software, or a combination of hardware and software.

The implantable pulse generator 805 is electrically and physically coupled to an intracardiac lead system 810. Portions of the intracardiac lead system 810 are shown inserted into the patient's heart 890. The intracardiac lead system 810 includes one or more electrodes configured to sense electrical cardiac activity of the heart, deliver electrical stimulation to the heart, sense the patient's transthoracic impedance, and/or sense other physiological parameters, e.g., cardiac chamber pressure or temperature. Portions of the housing 801 of the pulse generator 805 may optionally serve as a can electrode. An indifferent electrode 821 may be provided on a header of the pulse generator 805.

Communications circuitry is disposed within the housing 801, facilitating communication between the pulse generator 805 and an external device, such as an external diagnostic and/or therapy device, programmer, portable communicator, and/or networked advanced patient management system. The communications circuitry can also facilitate unidirectional or bidirectional communication with one or more implanted, external, cutaneous, or subcutaneous physiologic or non-physiologic sensors, patient-input devices and/or information systems (e.g., EMG sensor, EOG sensor, EEG sensor, blood pressure sensor, blood gas (oxygen and/or carbon dioxide) saturation sensor, accelerometer, microphone, impedance sensor, intrathoracic pressure sensor, left ventricular transmural pressure, heart rate sensor, hemodynamics sensor).

The pulse generator 805 may incorporate an EMG sensor 820 disposed on the housing 801 of the pulse generator 805. The EMG sensor may be configured, for example, to sense myopotentials of the patient's skeletal muscle in the pectoral region. Myopotential sensing may be used in connection with sleep disorders associated with involuntary limb movement.

The pulse generator 805 may further include a sensor configured to detect patient motion. The motion detector may be implemented as an accelerometer positioned in or on the housing 801 of the pulse generator 805. If the motion detector is implemented as an accelerometer, the motion detector may also provide acoustic information, e.g. rales, coughing, S1-S4 heart sounds, cardiac murmurs, and other acoustic information.

The lead system 810 of the CRM device 800 may incorporate a transthoracic impedance sensor that may be used to acquire the patient's cardiac output, or other physiological conditions related to the patient's sleep disorder(s). Other sensors, such as pressure and/or heart rate sensors, may also be used to determine a patient's cardiac output. For example, cardiac output may be computed as SV·PP, where SV is a measure of stroke volume and PP is a measure of pulse pressure. According to another approach, cardiac output may be computed as SV·HR, where SV is a measure of stroke volume and HR is a measure of heart rate.

The transthoracic impedance sensor may include, for example, one or more intracardiac electrodes 840, 842, 851-855, 863 positioned in one or more chambers of the heart 890. The intracardiac electrodes 841, 842, 851-855, 861, 863 may be coupled to impedance drive/sense circuitry 830 positioned within the housing of the pulse generator 805.

The impedance signal may also be used to detect the patient's respiration waveform and/or other physiological changes that produce a change in impedance, including pulmonary edema, heart size, cardiac pump function, etc. Respiratory and/or cardiac electrical therapy to treat disordered breathing or symptoms of same may be selected and/or altered on the basis of the patient's heart condition as sensed by impedance.

In one example, the transthoracic impedance may be used to detect the patient's respiratory waveform, examples of which are shown in FIGS. 8-11. A voltage signal developed at the impedance sense electrode 852, illustrated in FIG. 8, is proportional to the patient's transthoracic impedance and represents the patient's respiration waveform. The transthoracic impedance increases during respiratory inspiration and decreases during respiratory expiration. The transthoracic impedance may be used to determine the amount of air moved in one breath, denoted the tidal volume and/or the amount of air moved per minute, denoted the minute ventilation. A normal “at rest” respiration pattern, e.g., during non-REM sleep, includes regular, rhythmic inspiration—expiration cycles without substantial interruptions, as indicated in FIG. 8.

Returning to FIG. 13, the lead system 810 may include one or more cardiac pace/sense electrodes 851-855 positioned in, on, or about one or more heart chambers for sensing electrical signals from the patient's heart 890 and/or delivering pacing pulses to the heart 890. The intracardiac sense/pace electrodes 851-855, such as those illustrated in FIG. 13, may be used to sense and/or pace one or more chambers of the heart, including the left ventricle, the right ventricle, the left atrium and/or the right atrium. The lead system 810 may include one or more defibrillation electrodes 841, 842 for delivering defibrillation/cardioversion shocks to the heart. The lead system 810 may include one or more extracardiac leads 808 having electrodes, e.g., an epicardial electrode 818, positioned at locations outside the heart for sensing and/or pacing one or more heart chambers.

The pulse generator 805 may include circuitry for detecting cardiac arrhythmias and/or for controlling pacing or defibrillation therapy in the form of electrical stimulation pulses or shocks delivered to the heart through the lead system 810. As was discussed previously, CVHR detection circuitry 836 and sleep disorder diagnostic circuitry 835 may be housed within the housing 801 of the pulse generator 805. CVHR detection circuitry 836 and/or sleep disorder diagnostic circuitry 835 may be coupled to various sensors, including the transthoracic impedance sensor 830, EMG sensor 820, EEG sensor, EOG sensor, electrocardiogram sensors, nerve activity sensors, and/or other sensors capable of sensing physiological signals useful for CVHR and sleep disorder detection.

The sleep disorder diagnostic circuitry 835 may be coupled to a sleep disorder detector configured to detect sleep disorders such as disordered breathing, and/or movement disorders. An arousal detector and a sleep disorder detector may be coupled to a processor that may use information from the arousal detector and the sleep disorder detector to associate sleep disorder events with arousal events. The processor may trend the sleep disorder events and/or arousal events, associate the sleep disorder events with arousal events, and/or use the detection of the arousal events and/or the sleep disorder events for a variety of diagnostic purposes. The sleep disorder detector and/or the processor may also be configured as a component of the pulse generator 805 and may be positioned within the pulse generator housing 801. In one embodiment, information about the sleep disorder events and/or arousal events and CVHR may be used to adjust and titrate therapy delivered by the CRM device 800 and/or other therapy device.

A diagnostic and/or therapy device of the present invention may be used within the structure of an advanced patient management (APM) medical system. Advanced patient management systems may allow physicians to remotely and automatically monitor cardiac and respiratory functions, as well as other patient conditions. In one example, implantable cardiac rhythm management systems, such as cardiac pacemakers, defibrillators, and resynchronization devices, may be equipped with various telecommunications and information technologies that enable real-time data collection, diagnosis, and treatment of the patient. Various embodiments described herein may be used in connection with advanced patient management. Methods, structures, and/or techniques described herein, which may be adapted to provide for remote patient/device monitoring, trending, diagnosis, therapy, or other APM related methodologies, may incorporate features of one or more of the following references: U.S. Pat. Nos. 6,221,011; 6,270,457; 6,277,072; 6,280,380; 6,312,378; 6,336,903; 6,358,203; 6,368,284; 6,398,728; and 6,440,066, which are hereby incorporated herein by reference.

Disordered breathing and CVHR detection and/or treatment methodologies of the present invention may be implemented in a wide variety of implantable devices, such as cardiac monitoring and stimulation devices. For example, detection and/or treatment methodologies of the present invention may be implemented in cardiac monitoring devices, such as those disclosed in commonly owned U.S. Pat. Nos. 5,313,953; 5,388,578; and 5,411,031, which are hereby incorporated herein by reference. Detection and/or treatment methodologies of the present invention may be implemented in pacemaker devices, such as those disclosed in commonly owned U.S. Pat. Nos. 4,562,841; 5,284,136; 5,376,106; 5,036,849; 5,540,727; 5,836,987; 6,044,298; and 6,055,454, which are hereby incorporated herein by reference.

Detection and/or treatment methodologies of the present invention may be implemented in implantable cardioverter/defibrillator (ICD) devices, such as those disclosed in commonly owned U.S. Pat. Nos. 5,133,353; 5,179,945; 5,314,459; 5,318,597; 5,620,466; and 5,662,688, which are hereby incorporated herein by reference. Detection and/or treatment methodologies of the present invention may be implemented in cardiac devices that provide for heart failure monitoring, diagnosis, and/or therapy, such as those disclosed in commonly owned U.S. patent application Ser. No. 10/270,035, filed Oct. 11, 2002, entitled “Timing Cycles for Synchronized Multisite Cardiac Pacing;” and U.S. Pat. Nos. 6,411,848; 6,285,907; 4,928,688; 6,459,929; 5,334,222; 6,026,320; 6,371,922; 6,597,951; 6,424,865; and 6,542,775, each of which is hereby incorporated herein by reference.

Detection and/or treatment methodologies of the present invention may be implemented in subcutaneous, non-intrathoracic devices, such as those disclosed in commonly owned, U.S. patent application Ser. No. 10/465,520 filed Jun. 19, 2003, entitled “Methods and Systems Involving Subcutaneous Electrode Positioning Relative to a Heart”; Ser. No. 10/795,126 filed Mar. 5, 2004, entitled “Wireless ECG In Implantable Devices”; and Ser. No. 10/738,608 filed Dec. 17, 2003, entitled “Noise Canceling Cardiac Electrodes,” which are hereby incorporated herein by reference.

CVHR treatment methodologies of the present invention may involve vagal nerve stimulation. For example, treatment methodologies of the present invention may be implemented in nerve stimulation devices, such as those disclosed in commonly owned U.S. Publication Nos. 20050149126; 20050149129; and 20050149131; and in U.S. Publication No. 20050131467, which are hereby incorporated herein by reference.

CVHR treatment methodologies of the present invention may involve muscle stimulation. For example, treatment methodologies of the present invention may be implemented in muscle stimulation devices, such as those disclosed in commonly owned U.S. Publication No. 20050080463 and in U.S. Publication Nos. 20050137648; 20050278001; and 20050283204, which are hereby incorporated herein by reference.

Various modifications and additions may be made to the embodiments discussed herein without departing from the scope of the present invention. Accordingly, the scope of the present invention should not be limited by the particular embodiments described above, but should be defined only by the claims set forth below and equivalents thereof.

What we claim is:

1. A method, comprising:
detecting, within a patient, repeated occurrences of sleep disordered breathing; and
detecting, within the patient and after detecting the repeated occurrences of sleep disordered breathing, changes of a parameter indicative of cyclic variation of heart rate (CVHR).

2. The method according to claim 1, wherein detecting the repeated occurrences of sleep disordered breathing comprises detecting variations of a respiratory signal indicative of sleep disordered breathing.

3. The method according to claim 1, wherein the parameter comprises heart rate.

4. The method according to claim 1, wherein the parameter comprises a surrogate signal for heart rate.

5. The method according to claim 1, wherein the parameter comprises blood gas saturation.

6. The method according to claim 1, wherein the parameter comprises blood pressure.

7. The method according to claim 1, wherein the parameter comprises a parameter indicative of patient hemodynamics.

8. The method according to claim 1, wherein the parameter comprises cardiac output.

9. The method according to claim 1, wherein the parameter comprises autonomic activity.

10. The method according to claim 1, wherein the parameter comprises intrathoracic pressure.

11. The method according to claim 1, wherein the parameter comprises left ventricular transmural pressure.

12. The method according to claim 1, wherein detecting changes of the parameter indicative of CVHR comprises determining a periodicity of the parameter changes.

13. The method according to claim 1, wherein changes of the parameter indicative of CVHR comprises determining a measure of stability of the parameter changes.

14. A method, comprising:
detecting repeated occurrences of sleep disordered breathing;
detecting, after detecting the repeated occurrences of sleep disordered breathing, changes of a parameter indicative of cyclic variation of heart rate (CVHR); and
delivering a therapy to mitigate the CVHR.

15. The method according to claim 14, wherein the therapy comprises one or more of a drug therapy, a muscle stimulation therapy, a vagal nerve stimulation therapy, and a respiratory therapy, and delivering the therapy comprises adjusting one or more therapy parameters to mitigate the CVHR.

16. The method according to claim 14, wherein the therapy is a cardiac pacing therapy, and delivering the cardiac pacing therapy comprises adjusting one or more pacing parameters to mitigate the CVHR.

17. The method according to claim 14, wherein the therapy is a cardiac pacing therapy, and delivering the cardiac pacing therapy comprises adjusting at least one of a pacing mode, a pacing rate, and a pacing pattern to mitigate the CVHR.

18. The method according to claim 14, further comprising:
determining that the therapy mitigates the CVHR; and
continuing the therapy or administering another therapy until the sleep disordered breathing is mitigated.

19. A system, comprising:
a housing configured for implantation in a patient;
a sensor system, comprising:
a first sensor arrangement configured to sense one or more signals indicative of sleep disordered breathing; and
a second sensor arrangement configured to sense a physiological signal that exhibits cyclical behavior due to repeated occurrences of sleep disordered breathing; and
a processor provided in the housing and coupled to the sensor system, the processor configured to detect repeated occurrences of sleep disordered breathing using the one or more signals sensed by the first sensor arrangement, and to detect changes of the physiological signal indicative of cyclic variation of heart rate (CVHR).

20. The system according to claim 19, wherein the first sensor arrangement comprises a transthoracic impedance sensor.

21. The system according to claim 19, wherein the second sensor arrangement comprises at least one of an ECG sensor, blood pressure sensor, neural activity sensor, autonomic activity sensor, EMG sensor, EEG sensor, EOG sensor, photoplethysmography sensor, blood gas saturation sensor, intracardiac pressure sensor, and left ventricular transmural sensor.

22. A system, comprising:
a housing configured for implantation in a patient;
a pulse generator provided in the housing and coupled to a cardiac lead system;
a sensor system, comprising:
a first sensor arrangement configured to sense one or more signals indicative of sleep disordered breathing; and
a second sensor arrangement configured to sense a physiological signal that exhibits cyclical behavior due to repeated occurrences of sleep disordered breathing; and
a processor provided in the housing and coupled to the sensor system and the pulse generator, the processor configured to detect repeated occurrences of sleep disordered breathing using the one or more signals sensed by the first sensor arrangement, detect changes of the physiological signal indicative of cyclic variation of heart rate (CVHR), and deliver a cardiac electrical therapy to mitigate the CVHR.

23. The system according to claim 22, wherein one or both of the first and second sensor arrangements are supported by the cardiac lead system.

24. The system according to claim 22, wherein the processor is configured to adjust one or more pacing parameters of the cardiac electrical therapy to mitigate the CVHR.

25. The system according to claim 22, wherein the processor is configured to adjust at least one of a pacing mode, a pacing rate, and a pacing pattern to mitigate the CVHR.

26. The system according to claim 22, wherein the processor is configured to deliver or adjust a therapy to mitigate the sleep disordered breathing.
